(19) United States
(12) Patent Application Publication
(10) Pub. No.: US 2022/0409194 A1
(43) Pub. Date: Dec. 29, 2022

(54) LIGATION RELEASE DEVICE

(71) Applicant: MICRO-TECH (NANJING) CO., LTD., Nanjing (CN)

(72) Inventors: Zhou Yu, Nanjing, Jiangsu (CN); Zhi Tang, Nanjing, Jiangsu (CN); Changqing Li, Nangjing, Jiangsu (CN)

(73) Assignee: MICRO-TECH (NANJING) CO., LTD., Nanjing (CN)

(21) Appl. No.: 17/756,675

(22) PCT Filed: Apr. 30, 2020
(86) PCT No.: PCT/CN2020/087990

§ 371 Date: May 31, 2022

(30) Foreign Application Priority Data
Dec. 31, 2019 (CN) 201922466854.X

(51) Int. Cl. A61B 17/00; A61B 90/30

(52) U.S. Cl. CPC A61B 17/00234; A61B 90/30; A61B 2017/00358; A61B 2017/306

(57) ABSTRACT

The present application provides a ligation release device, comprising: a handle (1), an operating component (2), a suction component (3), a release component (4) and an illumination component (5), wherein the illumination component (5) is arranged in the handle (1) and comprises a light source (51), a wire (52) and a control switch (53), and the control switch (53) is connected to the light source (51) by means of the wire (52). In practical applications, the light source (51) can be turned on by operating the control switch (53), thereby illuminating a ligation area at the distal end of the handle (1), eliminating shadow influence and facilitating the implementation of ligation operation. The ligation release device can improve the ease and accuracy of surgical operation without an external auxiliary apparatus.

### CROSS-REFERENCE TO RELATED APPLICATION

The present application claims priority to Chinese Patent Application No. 201922466854. X, entitled “Ligation Release Device”, filed with the China Patent Office on Dec. 31, 2019, the entire disclosure of which is incorporated herein by reference.

### FIELD OF THE PRESENT DISCLOSURE

This application relates to the technical field of medical instrument, and more particularly to a ligation release device.

### BACKGROUND OF THE PRESENT DISCLOSURE

Ligation loop is a medical instrument used to ligate lesion tissue in minimally invasive surgery for hemorrhoids to carry out surgery measures such as resection of lesion tissue. A typical ligation loop is of a ring-like structure and can be made from a material having elasticity. During a surgery, the ligation loop can be propped open and controlled to move to the root of the lesion tissue. The ligation loop is then released, so as to be tightened to the root of the lesion tissue, which reduces tissue movement during the surgery procedure and surgery bleeding.

To facilitate ligation of the ligation loop to the lesion tissue root, the ligation loop can be released using a ligation loop release gun. The ligation loop release gun includes a gun-shaped handle, and an operating mechanism and a release mechanism provided on the handle. A member capable of generating negative pressure is provided inside or outside a release component. The operating mechanism controls the negative pressure component to generate negative pressure through a linkage relationship within the handle to draw lesion tissue into the release mechanism through the action of negative pressure. The ligation loop is then released through the operating mechanism to ligate the lesion tissue.

However, the lesion tissue of some patients may not be on the surface of the organ, and additional lighting is required during surgery because a visual field to observe the sucked lesion tissue is not provided during the release of the ligation loop. In addition, because of the gun-shaped handle structure of the ligation loop release gun, it is easy to generate shadow at the surgery region, and therefore it is inconvenient to ligate the lesion tissue within the ligation loop.

### SUMMARY OF THE PRESENT DISCLOSURE

The present application provides a ligation release device to solve the problem that a conventional ligation release device easily generates shadow at a surgery region.

The present application provides a ligation release device comprising: a handle, an operating component, a suction component, a release component, and an illumination component; wherein the operating component is provided on the handle; the suction component is provided in the handle, and the suction component is connected to the operating component to control the distal end at the handle to generate a negative pressure to suck the lesion tissue; and the release component is provided at a distal end of the handle to control the release component to release the ligation loop.

The illumination component is provided in the handle, and includes a light source, a wire and a control switch; wherein the light source is provided at a distal end of the handle, the control switch is provided at a proximal end of the handle, and the control switch connects the light source via the wire so as to form an illumination region at the distal end of the handle.

Alternatively, the release component comprises an inner sleeve, an outer sleeve and a transparent cap; wherein the transparent cap is a transparent cylindrical structure provided at a distal end of the inner sleeve, the ligation loop is provided on an outer circumferential wall of the transparent cap, and the outer sleeve engages an external wall of the inner sleeve via thread for releasing the ligation loop.

Alternatively, the light source is provided on the inner wall of the inner sleeve or on a proximal end of the transparent cap.

Alternatively, the light source is a line light source, and the light source is arranged axially on the inner wall of the inner sleeve; alternatively, the light source is arranged in an axial ring on the inner wall of the inner sleeve.

Alternatively, the light source is one or more uniformly arranged point light source; and the light source is provided at the proximal end of the transparent cap.

Alternatively, the ligation release device further comprises a viewing assembly; wherein the viewing assembly comprises one or more lens, and the viewing assembly is provided within the inner sleeve.

Alternatively, the suction component comprises a conduit and a negative pressure cylinder, and the negative pressure cylinder is connected to an inner cavity of the release component through the conduit.

Alternatively, the suction component further comprises a piston and a spring provided in the negative pressure cylinder, wherein one end of the piston is connected to the spring and the other end is connected to the operating component.

Alternatively, the conduit is connected to the transparent cap of the release component, the transparent cap is a cylinder with one side open, and the conduit is provided on the inner wall of the inner sleeve of the release component.

Alternatively, the handle comprises a grip portion, and the control switch is provided on the grip portion of the handle.

The present application provides a ligation release device comprising: a handle, an operating component, a suction component, a release component and an illumination component, wherein the illumination component is arranged in the handle and comprises a light source, a wire and a control switch, and the control switch is connected to the light source by the wire. In practical applications, the light source can be turned on by operating the control switch, thereby illuminating a ligation area at the distal end of the handle, eliminating shadow influence and facilitating the implementation of ligation operation. The ligation release device provided by the present application can improve the ease and accuracy of surgical operation without an external auxiliary apparatus.

### BRIEF DESCRIPTION OF THE DRAWINGS

In order to illustrate the technical solution of the present application more clearly, the drawings required to be used in the embodiment will be briefly described below, and it is obvious for a person skilled in the art to obtain other drawings without creative efforts.

FIG. 1 is a schematic structural diagram of a ligation release device of the present application;

FIG. 2 is a schematic structural diagram of the release component of the present application;

FIG. 3 is a schematic structural diagram of the suction component of the present application;

FIG. 4 is a schematic diagram of the overall structure of a ligation release device of the present application;

FIG. 5 is a schematic diagram of an exploded structure of ligation release device with viewing assembly in the present application.

### LIST OF THE REFERENCE NUMERALS IN THE DRAWINGS

1 handle
2 operating component
3 suction component
31 conduit
32 negative pressure cylinder
33 piston
34 spring
4 release component
41 inner sleeve
42 outer sleeve
43 transparent cap
5 illumination component
51 light source
52 wire
53 control switch
54 power supply
6 ligation loop
7 viewing assembly

### DESCRIPTION OF THE EMBODIMENTS

In the technical solution provided in the present application, for the convenience of description, an end of the whole device proximate to the patient's body is called a distal end, and this end is mainly used for a surgery on tissues; and an end proximate to a physician (surgeon) is called a proximal end, which is mainly used for operation by the surgeon. The proximal end and the distal end may not only refer to an orientation of the device as a whole, but may also refer to the orientation of individual components, that is to say, in the present application, unless otherwise specified, the distal end referred to by each component refers to an end proximate to the side of the patient, and the proximal end referred to by each component refers to an end proximate to the doctor.

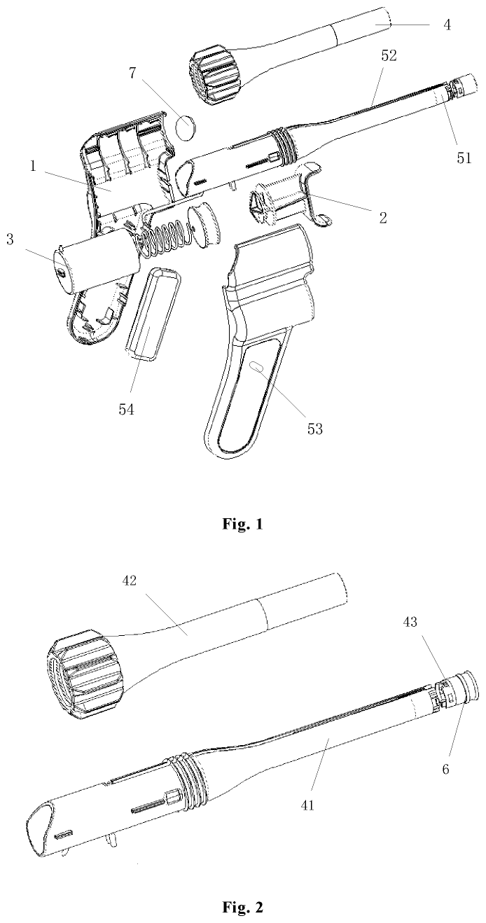

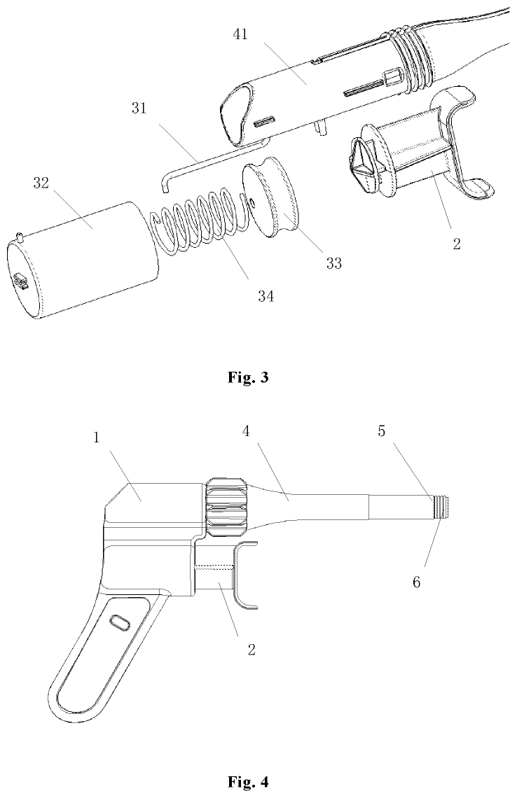

With reference to FIG. 1, a structural schematic diagram of a ligation release device of the present application is shown. It can be seen from FIG. 1 that the ligation release device provided in the present application can be applied to polyp excision, hemorrhoid and other surgeries. The device comprises: a handle 1, an operating component 2, a suction component 3, a release component 4, and an illumination component 5. As shown in FIG. 4, the handle 1 is held by a surgeon, and may be a pistol-like structure having an L-shape as a whole. The handle 1 may be of a hollow structure, including a grip portion and a fixed portion. The grip portion is used for an operator to hold, and the fixed portion is used for mounting and fixing other components. In order to reduce the overall weight and facilitate the operator to hold by one hand, the handle 1 can be made of two plastic bodies which are fastened to each other through an injection molding process. The injection molding material may be polyethylene (PE), polyvinyl chloride (PVC), polypropylene (PP), ABS plastic, etc.

The operating component 2 is provided on the handle 1 for the surgeon to carry out an operation, such as pressing, releasing, etc., so as to control the suction component 3 to generate negative pressure. The operating component 2 can be provided in a region closer to the holding position of the surgeon so that the surgeon can manipulate the operating component 2 by holding an index finger or a middle finger of the hand while holding the handle 1.

The suction component 3 is provided in the handle 1, and the suction component 3 is connected to the operating component 2. In an actual surgery, the operator can release the air in the suction component 3 by pressing, then move the whole device to where the patient is located, and then carry out a release operation, so that the negative pressure is generated in the suction component 3 and the lesion tissue of the distal end of handle 1 is sucked into the surgery region of the device.

The release component 4 is provided at a distal end of the handle 1 to release a ligation loop 6. The ligation loop 6 may be a circular ring made from an elastic material such as rubber. In actual applications, the ligation loop 6 can be ligated to the root of the lesion tissue through elasticity so that when the lesion tissue is resected, the tissue can be tightened to achieve hemostasis.

The illumination component 5 is provided in the handle 1 to illuminate the surgery region during surgery. The illumination component 5 comprises: a light source 51, a wire 52 and a control switch 53. The light source 51 may be a LED (light emitting diode) light source for converting an electrical signal to an optical signal. The control switch 53 may be a pressing-type or contact-type switch, and the wire 52 may be a copper wire or a row of wires. Obviously, if illumination is to be provided in the surgery region, it is also necessary to provide a power supply 54 to supply power for the light source 51. The power supply 54 may be a battery provided within the handle 1 that delivers electrical energy to the light source 51 through the wire 52 and the control switch 53 on the wire 52.

To facilitate illumination of the surgery region, the light source 51 is provided at the distal end of the handle 1, i.e., a location proximate to the surgery region, to provide the surgery region with greater brightness when turned on. The control switch 53 may be provided at any location of the handle 1, for example, at a location proximate to the handle proximal end, and may further be provided at a holding region proximate to the operator, so that the operator can directly carry out a turning on or off operation such as pressing. The control switch 53 is connected to the light source 51 through the wire 52 to control the turning on and off of the light source 51.

It can be seen from the above technical solution that the present application provides a ligation release device, comprising: a handle 1, an operating component 2, a suction component 3, a release component 4 and an illumination component 5, wherein the illumination component 5 is arranged in the handle 1 and comprises a light source 51, a wire 52 and a control switch 53, and the control switch 53 is connected to the light source 51 by means of the wire 52. In actual applications, the light source 51 can be turned on by operating the control switch 53, thereby illuminating a ligation area at the distal end of the handle 1, eliminating shadow influence and facilitating the implementation of ligation operation. The ligation release device provided by the present application can improve the ease and accuracy of surgical operation without an external auxiliary apparatus.

As shown in FIG. 2, in some embodiments of the present application, the release component 4 comprises: an inner sleeve 41, an outer sleeve 42 and a transparent cap 43. The transparent cap 43 is a transparent cylindrical structure provided at a distal end of the inner sleeve 41. Transparent cap 43 can be made from transparent material such as transparent resin, acryl, glass, etc., by an injection molding process. Since the transparent cap 43 is a region in direct contact with the lesion tissue, the tissue suction can be directly observed through transparent material to facilitate the surgery. The outer circumferential wall of the transparent cap 43 is provided with a ligation loop 6. The outer sleeve 42 engages the external wall of the inner sleeve 41 via thread, for releasing the ligation loop 6.

In actual applications, the operator first sets the ligation loop 6 on the external wall of the transparent cap 43. After generating negative pressure in the handle 1 by controlling an operating component, the lesion tissue is sucked into the transparent cap 43 or the inner sleeve 41. At this time, by rotating the outer sleeve 42, the outer sleeve 42 moves toward the distal end (surgery region) under the action of the threaded connection. When the outer sleeve 42 contacts the ligation loop 6, the ligation loop 6 can be pushed to also move toward the distal end. As the ligation loop 6 moves, the ligation loop 6 will ligate the root of the lesion tissue after being detached from the transparent cap 43 to complete the ligation operation.

It can be seen that in the present embodiment, the conduit 31 of the suction component 3 and the wire 52 of the illumination component 5 can be guided inside the cylinder by the inner sleeve 41. A cavity can also be formed inside the inner sleeve 41 and can be used for the operator to observe the region of the sucked tissue from the direction of the proximal end of the handle 1, so as to adjust the suction angle or the suction location of the lesion tissue during the surgery.

In order to generate negative pressure during suction, as shown in FIG. 3, in some embodiments of the present application, the suction component 3 comprises a conduit 31 and a negative pressure cylinder 32. The negative pressure cylinder 32 is connected to an inner cavity of the release component 4 via the conduit 31, i.e., connected to the inner cavity of the inner sleeve 41 or connected to the inner cavity of the transparent cap 43 via the conduit 31. In actual applications, the negative pressure cylinder 32 may drive a change in air pressure within the conduit 31 by compression or expansion to draw the air out of the inner sleeve 41 or the transparent cap 43 to form a negative pressure. When an end of the release component 4 is in contact with human tissue, lesion tissue can be sucked into the transparent cap 43 through negative pressure action.

As an alternative implementation, the suction component 3 further comprises a piston 33 and a spring 34 provided within the negative pressure cylinder 32 for evacuating air from the negative pressure cylinder 32 to form a negative pressure. In actual applications, the piston 33 is connected at one end to the spring 34 and at the other end to an operating component 2.

The operating component 2 can be in the form of a key. During surgery, the doctor first presses the operating component 2 to the end position to move the piston 33 to one side of the negative pressure cylinder 32, and then the spring 34 will be compressed. The transparent cap 43 is then aligned with the lesion tissue and compressed, then the operating component 2 is released. Under the action of the spring 34, the piston 33 and the operating component 2 are reset to form the negative pressure. The resulting negative pressure is then conducted through the conduit 31 to the transparent cap 43, thereby attracting the lesion tissue into the transparent cap 43.

Therefore, in some embodiments of the present application, the conduit 31 is connected to the transparent cap 43 of the release component 4; the transparent cap 43 is a cylinder with one side open; and the conduit 31 is provided on an inner wall of an inner sleeve 41 of a release component 4. The region sucking lesion tissue can be found in either the transparent cap 43 or the inner sleeve 41. If the suction region is in the transparent cap 43, it is required that the transparent cap 43 is a cylinder-shaped structure having an open end, and such a structure can facilitate the formation of the negative pressure, so that the cross-sectional area of the transparent cap 43 can be increased, the volume of the suction region can be increased, and the lesion tissue having a larger surface area can be sucked. If the suction region is provided in the inner sleeve 41, the inner sleeve 41 is of a cylindrical structure in its whole and communicates with the transparent cap 43, and such a structure can have a larger region capable of accommodating the lesion tissue in the inner sleeve 41, so that a larger volume of the lesion tissue can be sucked.

In the technical solution provided in the present application, a surgeon can observe the suction of the lesion tissue outside of the release component 4, and can also observe the suction of the lesion tissue through the inner sleeve 41.

For easy observation, the surgery region may be illuminated by a light source 51, and therefore, the light source 51 may be provided on the inner wall of the inner sleeve 41 or on the proximal end of the transparent cap 43 to illuminate the surgery region by illumination. The light source 51 may be a line light source. The line light source can be provided by uniformly arranging a plurality of LEDs and supplemented with lamp strips formed by fluorescent substance. The light source 51 is arranged axially on the inner wall of the inner sleeve 41, so that the entire inner sleeve 41 can be illuminated. Thus, after the lesion tissue is sucked in the inner sleeve 41, blocking of the light source 51 by the tissue is avoided. The light source 51 may also be arranged in an axial ring on the inner wall of the inner sleeve 41 to increase the illumination intensity of the surgery region.

In some embodiments of the present application, the light source 51 is one or more uniformly arranged point light source, and the light source 51 is provided at the proximal end of the transparent cap 43. In actual applications, if the transparent cap 43 is a cylindrical structure with one end open, the light source 51 can be provided at the end of the transparent cap 43 that is blocked and the irradiating direction faces the region sucking the tissue (i.e., a distal end). The light source 51 may be one or more point light source evenly distributed along the annular direction to avoid obstructing the field of view as much as possible.

In addition, the conduit 31 is provided on the inner wall of the inner sleeve 41 to prevent the conduit 31 from obstructing the operator's view field, which facilitates the observation during the surgery process. In actual applications, a groove can be provided on the inner wall of the inner sleeve 41 and the conduit 31 can be provided in the groove. The depth of the groove may be slightly greater than the outer diameter of the conduit 31, so as to reduce the distance that the conduit 31 protrudes from the inner wall of the inner sleeve 41, and to avoid obstructing the view field.

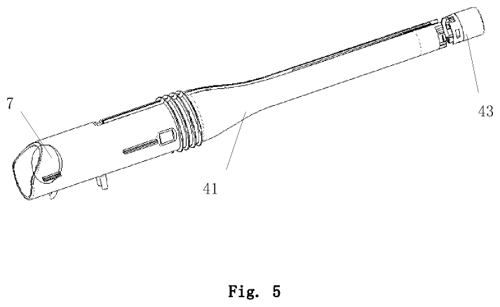

In some embodiments of the present application, as shown in FIG. 5, the ligation release device further comprises a viewing assembly 7; the viewing assembly 7 comprises one or more lens; and the viewing assembly 7 is provided in the inner sleeve 41. The viewing assembly 7 may amplify the surgery region by refraction of light by one or more lens to facilitate viewing of the surgery region by the surgeon.

It can be seen that in the above embodiments, the illumination component 5 is mainly composed of a light source 51, a wire 52 and a control switch 53. The light source 51 is located at the transparent cap 43 of distal end of the inner sleeve 41, and the light source 51 can be lit up by operating the control switch 53, so that the surgery region in the transparent cap 43 can be illuminated. For ease of operation, the control switch 53 may be provided within the grip region of the handle 1 so that the operator can carry out the operation by hand to light up or turn off the light source 51. In a possible embodiment, the control switch 53 can also be provided on the operating component 2, and the light source 51 can be lit up at the same time when the operating component 2 is pressed, thereby reducing the additional surgery action and reducing the difficulty of surgery implementation.

It can be seen from the above technical solution that the present application provides a ligation release device, comprising: a handle 1, an operating component 2, a suction component 3, a release component 4 and an illumination component 5, wherein the illumination component 5 is arranged in the handle 1 and comprises a light source 51, a wire 52 and a control switch 53, and the control switch 53 is connected to the light source 51 by the wire 52. In practical applications, the light source 51 can be turned on by operating the control switch 53, thereby illuminating a ligation area at the distal end of the handle 1, eliminating shadow influence and facilitating the implementation of ligation operation. The ligation release device provided by the present application can improve the ease and accuracy of surgical operation without an external auxiliary apparatus.

The similar parts between the embodiments provided in this application can be referred to each other. The embodiments provided above is only a few examples under the general concept of the present application, and does not constitute a limitation to the scope of the present application. Any other embodiments that can be extended from the solution of the present application by the person skilled in the art without inventive efforts will be within the scope of protection of the present application.

1. A ligation release device, comprising:
a handle;
an operating component provided on the handle;
a suction component provided in the handle, the suction component being connected to the operating component to control a distal end at the handle to generate a negative pressure to suck lesion tissue; and
a release component provided at a distal end of the handle to release a ligation loop;
characterized by further comprising an illumination component;
wherein the illumination component comprises a light source, a wire and a control switch; the control switch connects the light source through the wire to form an illumination region at the distal end of the handle.

2. The ligation release device according to claim 1, characterized in that the release component comprises an inner sleeve, an outer sleeve and a transparent cap;
the transparent cap is a transparent cylindrical structure provided at a distal end of the inner sleeve; the ligation loop is provided on the outer circumferential wall of the transparent cap; and the outer sleeve engages the external wall of the inner sleeve via thread for releasing the ligation loop.

3. The ligation release device according to claim 2, characterized in that the light source is provided on the inner wall of the inner sleeve or on a proximal end of the transparent cap.

4. The ligation release device according to claim 3, characterized in that the light source is a line light source, and the light source is arranged axially on the inner wall of the inner sleeve; or
the light source is provided in an axial ring on the inner wall of the inner sleeve.

5. The ligation release device according to claim 3, characterized in that the light source is one or more uniformly arranged point light source; and the light source is provided at the proximal end of the transparent cap.

6. The ligation release device according to claim 3, characterized in that the ligation release device further comprises a viewing assembly; wherein the viewing assembly comprises one or more lens, and the viewing assembly is provided within the inner sleeve.

7. The ligation release device according to claim 1, characterized in that the suction component comprises a conduit and a negative pressure cylinder, and the negative pressure cylinder is connected to an inner cavity of the release component through the conduit.

8. The ligation release device according to claim 7, characterized in that the suction component further comprises a piston and a spring provided in the negative pressure cylinder, wherein one end of the piston is connected to the spring and the other end is connected to the operating component.

9. The ligation release device according to claim 7, characterized in that the conduit is connected to the transparent cap of the release component; the transparent cap is a cylinder with one side open; and the conduit is provided on the inner wall of the inner sleeve of the release component.

10. The ligation release device according to claim 1, characterized in that the handle comprises a grip portion, and the control switch is provided on the grip portion of the handle.
